(19) United States
(12) Patent Application Publication
(10) Pub. No.: US 2009/0291489 A1
(43) Pub. Date: Nov. 26, 2009

(54) Disposal apparatus of food wastes

(76) Inventors: Bok Gyeong Kim, Suwon-si (KR); Jin Hyun Shon, Seongnam-si (KR); Byeong Sik Choi, Seoul (KR)

Correspondence Address: MCDERMOTT WILL & EMERY LLP, 600 13TH STREET, N.W., WASHINGTON, DC, 20005-3096, US

(21) Appl. No.: 12/153,718

(22) Filed: May 23, 2008

(51) Int. Cl. C12M 1/02

(52) U.S. Cl. 435/290.2

(57) ABSTRACT

Disclosed is a disposal apparatus of food wastes. The disposal apparatus includes a reactor for permitting the food wastes and microbes for decomposing the food wastes to be contained therein; an agitator mounted inside the reactor for mixing the food wastes and the microbes; an air inlet connected with the reactor for permitting air to flow into the reactor; and an air heater connected with the air inlet for heating the air flowing into the reactor.

### BACKGROUND OF THE INVENTION

1. Field of the Invention

The present invention relates to a disposal apparatus of food wastes.

2. Description of Related Art

Most of food wastes are generally landfilled. However, when the food wastes contain a relatively great amount of water such as in the case of the food wastes, a great amount of leachate may be created from the food wastes by landfilling. Also, the created leachate may disadvantageously act as contaminants in soil, water, and the like. In addition, the food wastes may be easily decayed in foods collection and transportation processes, which leads to cause problems such as generation of offensive odors and the like.

As another method for processing the food wastes, an incineration treatment method may be used. However, even in this case, incineration efficiency may be reduced due to the food wastes containing the relatively great amount of water, and accordingly a high cost may be required for the incineration treatment of the food wastes. In addition, there arise problems such as generating harmful substances like dioxin, and the like.

Thus, there is a need for a method for nature-friendly processing of the food wastes.

### SUMMARY OF THE INVENTION

An aspect of the present invention provides a disposal apparatus of food wastes which may nature-friendly decompose the food wastes.

An aspect of the present invention provides a disposal apparatus of food wastes which may effectively decompose the food wastes without reducing a moisture content of the food wastes.

According to an aspect of the present invention, there is provided a disposal apparatus of food wastes including: a reactor for permitting the food wastes and microbes for decomposing the food wastes to be contained therein; an agitator mounted inside the reactor for mixing the food wastes and the microbes; an air inlet connected with the reactor for permitting air to flow into the reactor; and an air heater connected with the air inlet for heating the air flowing into the reactor.

### BRIEF DESCRIPTION OF THE DRAWINGS

The above and other aspects of the present invention will become apparent and more readily appreciated from the following detailed description of certain exemplary embodiments of the invention, taken in conjunction with the accompanying drawings of which:

FIG. 1 is a cross-sectional view illustrating a disposal apparatus of food wastes according to an exemplary embodiment of the present invention.

### DETAILED DESCRIPTION OF EXEMPLARY EMBODIMENTS

Reference will now be made in detail to exemplary embodiments of the present invention, examples of which are illustrated in the accompanying drawings, wherein like reference numerals refer to the like elements throughout. The exemplary embodiments are described below in order to explain the present invention by referring to the figures.

A disposal apparatus of food wastes according to an exemplary embodiment of the present invention which may nature-friendly decompose the food wastes using microbes, unlike a conventional landfilling or incineration treatment scheme.

Here, the food wastes may be wastes generated when processing foods in houses, restaurants, industrial facilities, and the like, or wastes required to be processed after eating the foods. The food wastes may be food wastes generated from grains, meats, fishes, fruits, vegetables, various sources, and the like, however, the present invention is not limited thereto.

The microbes may function to decompose the food wastes, and types of the microbes are not limited in particular. For example, the microbes may be Bacillus smithi. Bacillus smithi is one of bacterium which is capable of decomposing the food wastes at a relatively high temperature environment of about 35° C. to 60° C., has a superior decomposition capacity for cellulose of a non-resolutive substance, and is capable of decomposing N-acetyl-glucosamine composing a cell wall. Bacillus smithi may be used alone or in a mixture with aerotropic microbes such as a yeast pertaining to ascomycetes. Alternatively, Bacillus smithi may be used in a mixture with well-known microbes capable of decomposing the food wastes. Thus, according to the present invention, types of the microbes are not limited as long as the microbes can decompose the food wastes.

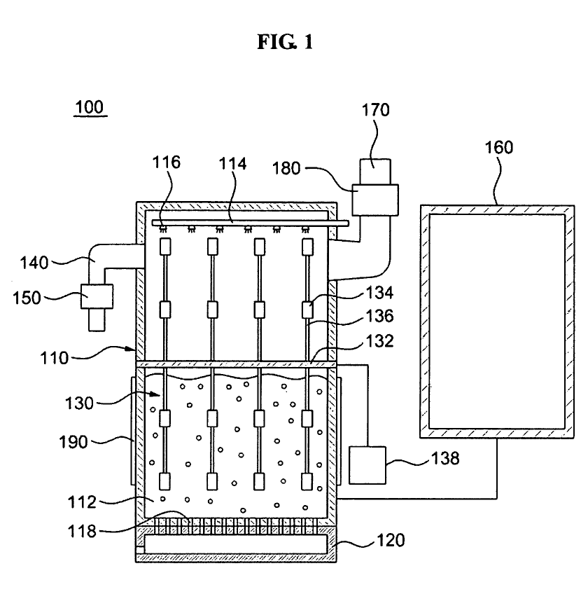

FIG. 1 is a cross-sectional view illustrating a disposal apparatus 100 of food wastes according to an exemplary embodiment of the present invention.

Referring to FIG. 1, the disposal apparatus 100 of food wastes according to the present exemplary embodiment of the present invention includes a reactor 110, an agitator 130, an air inlet 140, and an air heater 150. Also, the disposal apparatus 100 of food wastes according to the present exemplary embodiment of the invention may further include a pulverizer 160, a gas outlet 170, and a reactor heating unit 190.

Food wastes 112 and microbes for decomposing the food wastes 112 may be contained in the reactor 110. In addition, a small amount of a bulking agent may be contained in the reactor 110. Here, at least one from a group consisting of sawdust, chaff, leaf mold, peat moss, dried sphagnum moss, bark, and coconut peat may be selected for the bulking agent.

The reactor 110 may be formed in a drum-shape having an open top. A cover (not shown) capable of being opened and closed while covering an upper portion of the reactor 110 may be mounted on a vicinity of the open top. In this case, the cover is opened, and then the food wastes 112 are inserted into the reactor 110.

A plurality of water spraying nozzles 116 are mounted on the upper portion of the reactor 110 for spraying water supplied via a water supplying pipe 114 to an inside of the reactor 110. The plurality of water spraying nozzles 116 may spray the water to the inside of the reactor 110 to thereby clean the inside of the reactor 110. In addition, the plurality of water spraying nozzles 116 may spray the water to the inside of the reactor 110 to thereby contribute to decomposition of the food wastes 112. Specifically, the food wastes 112 may be decomposed into smaller particles by the sprayed water. Also, a gas generated when decomposing the food wastes 112 may be dissolved into the water sprayed through the plurality of water spraying nozzles 116, which leads to removal of offensive odors. In this manner, a reason for spraying water into the inside of the reactor 110 is that the disposal apparatus 100 according to the present exemplary embodiment of the invention is capable of processing the food wastes 112 even with a moisture content of about 85% or more.

The reactor 110 may include a leachate draining unit 118 mounted on a lower portion of the reactor 110 for draining a leachate generated within the reactor 110 to the outside. A leachate draining unit 118 mounted on a bottom surface of the reactor 110 may be formed in a net-shape having fine holes, however, since the leachate draining unit 118 may be formed in various shapes, the present invention is not limited by a structure of the leachate draining unit 118.

A discharging unit 120 may be mounted on a lower portion of the reactor 110. The discharging unit 120 may receive the leachate drained from the reactor 110 via the leachate draining unit 118, and discharge the received leachate to the outside. In this instance, the leachate discharged to the outside may preferably flow into a decontamination facility or a wastewater treatment facility.

The agitator 130 may be mounted inside the reactor 110 for mixing the food wastes 112 and the microbes. The agitator 130 may include a shaft 132 horizontally mounted within the reactor 110 and a plurality of stirring arms 136 connected with the shaft 132 and having a plurality of stirring wings 134. In this case, when the shaft 132 is rotated by a motor 138 connected with the shaft 132, the plurality of stirring arms 136 and the plurality of stirring wings 134 are accordingly rotated to thereby mix the food wastes 112 and the microbes. A rotation speed of the agitator 130 may be about 1,000 to 10,000 rpm, which vary depending on a kind or amount of foods, however, the present invention is not limited thereto.

The air inlet 140 may be mounted to be connected with the reactor 110 for permitting air to flow into the reactor 110. The air inlet 140 may be constructed to have a ring blower, so that the air is forced to flow into the reactor 110. An activity of aerotropic microbes may increase inside the reactor 110 due to the air, thereby promoting decomposition of foods.

The air heater 150 may be mounted to be connected with the air inlet 140 for heating the air flowing into the reactor 110. The air heater 150 may heat the air to about 30° C. to 60° C. to promote decomposition by the microbes, and thus the present invention is not limited by a specific configuration of the air heater 150.

The pulverizer 160 may be mounted to be connected with the reactor 110 for pulverizing the food wastes 112. Specifically, the pulverizer 160 may pulverize the food wastes 112 to have a particle size being appropriate for types of the food wastes 112 and the activity of the microbes. The food wastes 112 pulverized in the pulverizer 160 may be transported to the reactor 110 through a transportation screw (not shown). However, the disposal apparatus 100 of food wastes according to the present exemplary embodiment of the invention may not include the pulverizer 160. In this case, the food wastes 112 may not pass through the pulverizer 160 and may be directly transported to the inside of the reactor 110.

The gas outlet 170 may be mounted to be connected with the reactor 110 for exhausting a gas generated when the food wastes 112 are decomposed by the microbes. As a result, the gas outlet 170 may prevent an internal pressure of the reactor 110 from increasing. A gas processing unit 180 may be mounted to be connected with the gas outlet 170. The gas processing unit 180 may include an air filter, and remove offensive odors using the air filter.

The reactor heating unit 190 may be mounted on an outer peripheral surface of the reactor 110 for heating the reactor 110 in order to promote decomposition of the food wastes 112. The reactor heating unit 190 may be formed in a flat plate-shape and attached on the outer peripheral surface of the reactor 110, thereby directly heating the reactor 110. However, since a structure of the reactor heating unit 190 may vary depending on needed conditions and design environment, the present invention is not limited thereto.

Operations of the disposal apparatus 100 of food wastes according to the present exemplary embodiment of the invention will be herein described in detail. First, the food wastes 112 intended to be treated and the microbes for decomposing the food wastes 112 are contained within the reactor 110. In this instance, the bulking agent other than the food wastes 112 and the microbes may be further contained within the reactor 110. Next, the motor 138 is operated to drive the agitator 130. Then, the food wastes 112 and the microbes are mixed with each other within the reactor 110 to thereby decompose the food wastes 112. In this instance, a leachate generated within the reactor 110 may pass through the leachate draining unit 118 and the discharging unit 120 and discharged to the outside. When the above described-process for decomposing the food wastes 112 is completed, the food wastes 112 may be discharged to the outside of the reactor 110 and then the discharged food wastes may be scraped.

As described above, the disposal apparatus 100 of food wastes according to the present exemplary embodiment of the present invention may nature-friendly process the food wastes 112 using the microbes. Also, the disposal apparatus 100 of food wastes according to the present exemplary embodiment of the present invention may decompose the food wastes 112 having a relatively high moisture content, thereby omitting a process of reducing the moisture content before decomposition by the microbes, and also significantly reducing an amount of the bulking agent used for disposal of the food wastes 112. Also, the disposal apparatus 100 of food wastes according to the present exemplary embodiment of the present invention may significantly reduce a cost required for processing the food wastes 112, and more easily process the food wastes 112.

Hereinafter, the present invention will be described in detail by examples. It is to be understood, however, that these examples are for illustrative purpose only, and are not construed to limit the scope of the present invention.

### Example

### Preparation for Processing Food Wastes

Food wastes including boiled rice, vegetables (radish, cabbage, dropwort), and the like, Bacillus smithi, and coconut peat were contained inside the reactor of the disposal apparatus of food wastes according to an exemplary embodiment of the present invention, and thereby the preparation for processing the food wastes was completed. In this instance, as shown in the below Table 1, at least one of an amount of microbes, a moisture content, and an internal temperature of the reactor was varied, and thereby the preparation for processing the food wastes of Examples 1 to 3 and Comparative Examples 1 to 3 was completed.

TABLE 1

| | | | Amount | | |
|---|---|---|---|---|---|
| | | | of | | |
| | Amount of | Amount of | food | Moisture | Internal |
| | Microbes | bulking | wastes | content | temperature |
| | (ml) | agent (kg) | (kg) | (%) | of reactor |
| Example 1 | 15 | 2 | 63.4 | 84.7 | 44.5 |
| Example 2 | 15 | 2 | 63.4 | 75 | 44.5 |
| Example 3 | 15 | 2 | 63.4 | 40 | 44.5 |
| Comparative | | 2 | 63.4 | 40 | 44.5 |
| Example 1 | | | | | |
| Comparative | | 2 | 63.4 | 75.7 | 44.5 |
| Example 2 | | | | | |
| Comparative | | 2 | 63.4 | 40.7 | 44.5 |
| Example 3 | | | | | |

Estimation of Decomposition Capacity for Food Wastes Depending On Moisture Content

In order to estimate decomposition capacity for food wastes depending on a moisture content of the food wastes, the decomposition capacity for food wastes was estimated with respect to the Examples 1 to 3, and Comparative Examples 1 to 3.

The estimation of decomposition capacity for food wastes was performed right after weighing of the food wastes was inserted in the reactor, after 24 hours, after 48 hours, after one week, and after two weeks, respectively, and the results are shown in the below Table 2. Here, a speed of the agitator of the disposal apparatus of food wastes according to the present exemplary embodiment of the invention was maintained to be about 6,000 rpm for 24 hours

TABLE 2

| | Weight of food wastes | | | | |
|---|---|---|---|---|---|
| | Right | | | | |
| | after | | | | |
| | being | | | | |
| | inserted | After 24 | After 48 | After one | After two |
| | (kg) | hours (kg) | hours (kg) | week (kg) | weeks (kg) |
| Example 1 | 63.4 | 52.2 | 51.5 | 35 | 5.6 |
| Example 2 | 63.4 | 52.1 | 48.0 | 31 | 5.5 |
| Example 3 | 63.4 | 53.8 | 49.5 | 29.9 | 5.0 |
| Comparative | 63.4 | 59.2 | 56.5 | 43.3 | 33.1 |
| Example 1 | | | | | |
| Comparative | 63.4 | 58.4 | 54.7 | 39.9 | 28.6 |
| Example 2 | | | | | |
| Comparative | 63.4 | 58.2 | 53.8 | 38.3 | 13.5 |
| Example 3 | | | | | |

As can be seen in the above Table 2, when the microbes were inserted in the reactor, the decomposition capacity for the food wastes did not show much difference in Examples 1 to 3. Conversely, when the microbes were not inserted in the reactor, the decomposition capacity for the food wastes was significantly deteriorated along with an increase in the moisture content in Comparative Examples 1 to 3. In addition, the decomposition capacity for the food wastes of Examples 1 to 3 was significantly superior in comparison with that of Comparative Examples 1 to 3.

Estimation of Degree of Offensive Odor of Food Wastes

In order to see effects in removing offensive odors of food wastes, a degree of offensive odor was measured with respect to Examples 1 to 3 and Comparative Examples 1 to 3, respectively.

The estimation of the degree of the offensive odor was performed such that twenty sensory testers were selected, and then the degree of the offensive odor was estimated using a 7-point scalar scoring test {(significantly bad (1-point), mediocre (4-point), few or no (7-point), etc.}. The results are shown in the below Table 3.

TABLE 3

| | One week | Two weeks |
|---|---|---|
| Example 1 | 4.4 | 4.8 |
| Example 2 | 4.1 | 4.7 |
| Example 3 | 4.6 | 5.1 |
| Comparative Example 1 | 2.1 | 1.9 |
| Comparative Example 2 | 1.8 | 2.3 |
| Comparative Example 3 | 1.7 | 2.6 |

As can be seen in Table 3, in the case where the microbes were inserted in the reactor as Examples 1 to 3, the degree of the offensive odor was significantly less in comparison with the case where the microbes were not inserted in the reactor.

As described above, according to the present invention, the food wastes are decomposed using the microbes, thereby enabling to nature-friendly process the food wastes. In addition, even when the food wastes having a relatively high moisture content are inserted in the reactor, the food wastes are easily decomposed by the microbes, thereby significantly reducing a cost required for processing the food wastes, and more easily processing the food wastes.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. A disposal apparatus of food wastes, comprising:
a reactor for permitting the food wastes and microbes for decomposing the food wastes to be contained therein;
an agitator mounted inside the reactor for mixing the food wastes and the microbes;
an air inlet connected with the reactor for permitting air to flow into the reactor; and
an air heater connected with the air inlet for heating the air flowing into the reactor.

2. The disposal apparatus of food wastes of claim 1, further comprising:
a pulverizer connected with the reactor for pulverizing the food wastes.

3. The disposal apparatus of food wastes of claim 1, further comprising:
a gas outlet connected with the reactor for exhausting a gas generated when the food wastes are decomposed by the microbes.

4. The disposal apparatus of food wastes of claim 1, further comprising:
a reactor heating unit mounted on an outer peripheral surface of the reactor for heating the reactor in order to promote decomposition of the food wastes.

5. The disposal apparatus of food wastes of claim 4, wherein the reactor heating unit is formed in a flat plate-shape and attached on the outer peripheral surface of the reactor.

6. The disposal apparatus of food wastes of claim 1, wherein the reactor is formed in a drum-shape having an open top.

7. The disposal apparatus of food wastes of claim 1, further comprising:
a water spraying nozzle mounted on an upper portion of the reactor for spraying water to an inside of the reactor.

8. The disposal apparatus of food wastes of claim 1, wherein the reactor includes a leachate draining unit mounted on a lower portion of the reactor for draining a leachate generated within the reactor to the outside.

9. The disposal apparatus of food wastes of claim 8, further comprising:
a discharging unit mounted on a lower portion of the reactor for discharging the leachate drained through the leachate draining unit to the outside.

10. The disposal apparatus of food wastes of claim 1, wherein the agitator comprises:
a shaft horizontally mounted within the reactor; and
a plurality of stirring arms connected with the shaft and having a plurality of stirring wings.

11. The disposal apparatus of food wastes of claim 1, wherein the microbes comprises Bacillus smithi.
